(19) United States
(12) Patent Application Publication
(10) Pub. No.: US 2011/0008865 A1
(43) Pub. Date: Jan. 13, 2011

(54) INTEGRATED WASTE/HEAT RECYCLE SYSTEM

(75) Inventor: Olaf Nathan Lee, Centerville, MN (US)

Correspondence Address: PAULY, DEVRIES SMITH & DEFFNER, L.L.C., Plaza VII-Suite 3000, 45 South Seventh Street, MINNEAPOLIS, MN, 55402-1630, US

(73) Assignee: VISIAM, LLC, White Bear Lake, MN (US)

(21) Appl. No.: 12/816,438

(22) Filed: Jun. 16, 2010

Related U.S. Application Data
(60) Provisional application No. 61/187,463, filed on Jun. 16, 2009.

(51) Int. Cl. C12P 5/00; C12M 1/107; C12P 1/00; C12P 1/04

(52) U.S. Cl. 435/166; 435/290.1; 435/41; 435/170

(57) ABSTRACT

An integrated system and process for the treatment of organic fractions of municipal solid waste is described herein. The system and the process transform solid waste into fuel and energy. The integrated system and process comprise various different processes for pretreatment, sorting/separating, anaerobic digestion and conversion of biomass and gas to various gasseous, liquid and solid fuels and electricity.

### CROSS REFERENCE TO RELATED APPLICATION

This application claims the benefit of U.S. Provisional Patent Application Ser. No. 61/187,463, filed Jun. 16, 2009, which application is hereby incorporated by reference in its entirety.

### BACKGROUND OF THE INVENTION

The disposal of solid waste materials is a serious problem for public and private organizations. Recycling programs have been successful at using only a portion of this waste stream, whereas a good portion of the waste stream is either burned or left in landfills.

The amount of solid waste, particularly municipal solid waste, generated by individual households, businesses and governmental sites has increased significantly over time. Disposal of such waste materials has become more difficult. The inconvenience of waste disposal has increased along with the environmental impact of solid waste on land use, potable water, the atmosphere and the natural environment.

A large fraction of municipal solid waste (MSW) streams in the United States are comprised of natural organic compounds, including food and plant wastes. These organic fractions have low heat value and high moisture content, which normally make such waste streams undesirable for combustion in waste-to-energy (WTE) plants. But these properties are desirable in systems using anaerobic digestion to produce methane gas. The produced gas can be captured and used for energy cogeneration.

The use of anaerobic digestion on the organic fraction of municipal solid waste (OFMSW) streams reduces the volume of waste sent to landfills and thereby decreases emissions of greenhouse gases such as methane produced by waste decay. In addition, biogas generated by anaerobic digestion sites is used to produce electricity and heat that is sold to utilities and district heating facilities. A substantial need is seen to obtain value from waste while conserving or producing a net gain in energy.

### SUMMARY OF THE INVENTION

An integrated system and process for the treatment of organic and inorganic fractions of municipal solid waste is described herein. The system and the process transform solid waste into useful product streams, including fuel, and energy.

In an embodiment, the system comprises an integrated waste processing system that includes subsystems for pretreating municipal solid waste (MSW), separating and sorting the pretreated waste, anaerobic digestion of the separated organic fractions and subsystems for gas storage and cogeneration of energy. In an aspect, the subsystem for pretreating MSW includes one or more pressurized vessels for pretreating the solid waste by addition of heat and water. In an aspect, the subsystem for separating and sorting the pretreated waste includes a separator for separating the solid waste into an organic fraction and a recyclable materials fraction. In an aspect, the subsystem for anaerobic digestion includes a process for digestion of the organic fraction of municipal solid waste by thermophilic microorganisms. In an aspect, the anaerobic digestion system produces methane, carbon dioxide and compost materials. In an aspect, the waste processing system comprises a subsystem including a flare, if needed, and a low energy fuel reciprocating engine cogenerator which are used to process methane gas produced by anaerobic digestion of OFMSW. In an aspect, combustion of methane gas produces heat that is recovered to offset gas consumption in the integrated waste processing system.

In an embodiment, the process for treating municipal waste streams includes pretreating municipal solid waste, followed by sorting and separating the pretreated municipal solid waste into an organic fraction and a recyclable materials fraction. The organic fraction is then subjected to anaerobic digestion, and the products of the digestion are converted into fuel. In an aspect, pretreating municipal solid waste comprises introducing the waste stream into a rotary vessel, adding a quantity of water and reducing the pressure inside the vessel. The interior of the vessel is then heated and the pretreated solid waste is evacuated in a single stream for separation and sorting. In an aspect, separating and sorting the pretreated waste stream comprises separating the organic fines from the pretreated waste stream as the organic fraction and sorting the recyclable materials by type. In another aspect, anaerobic digestion of the organic fraction of the waste stream comprises contacting the organic fraction with thermophilic microorganisms thereby breaking down the organic fraction and converting it to biogas, i.e. a mixture of carbon dioxide and methane. In yet another aspect, the process comprises converting the methane gas into fuel using a low reciprocity engine cogenerator, and recovering waste heat from combustion of methane to offset gas consumption in the WTE plants.

It is to be understood that both the foregoing general description and the following detailed description are exemplary and explanatory only and are not restrictive of the invention, as claimed. The accompanying drawings, which are incorporated in and constitute a part of this specification, illustrate an example embodiment of the invention and together with the description, serve to explain the principles of the invention.

### BRIEF DESCRIPTION OF THE DRAWINGS

FIG. 1 is a diagram illustrating the integration of various subsystems into a single integrated system for energy cogeneration.

FIG. 2 is a diagram depicting plastics, ferrous and non-ferrous materials being conveyed to a single baler during separation and sorting of municipal solid waste.

FIG. 3 is a schematic representation of the process for conversion of municipal solid waste to fuel.

### DETAILED DESCRIPTION OF THE PREFERRED EMBODIMENT

The systems and methods described herein provide an integrated system and process for converting waste streams into valuable fuel and recyclable materials. This is accomplished in a straightforward method using energy-efficient, environmentally sound and cost-effective equipment to process MSW, especially the OFMSW, to produce a clean fuel and recyclable materials stream.

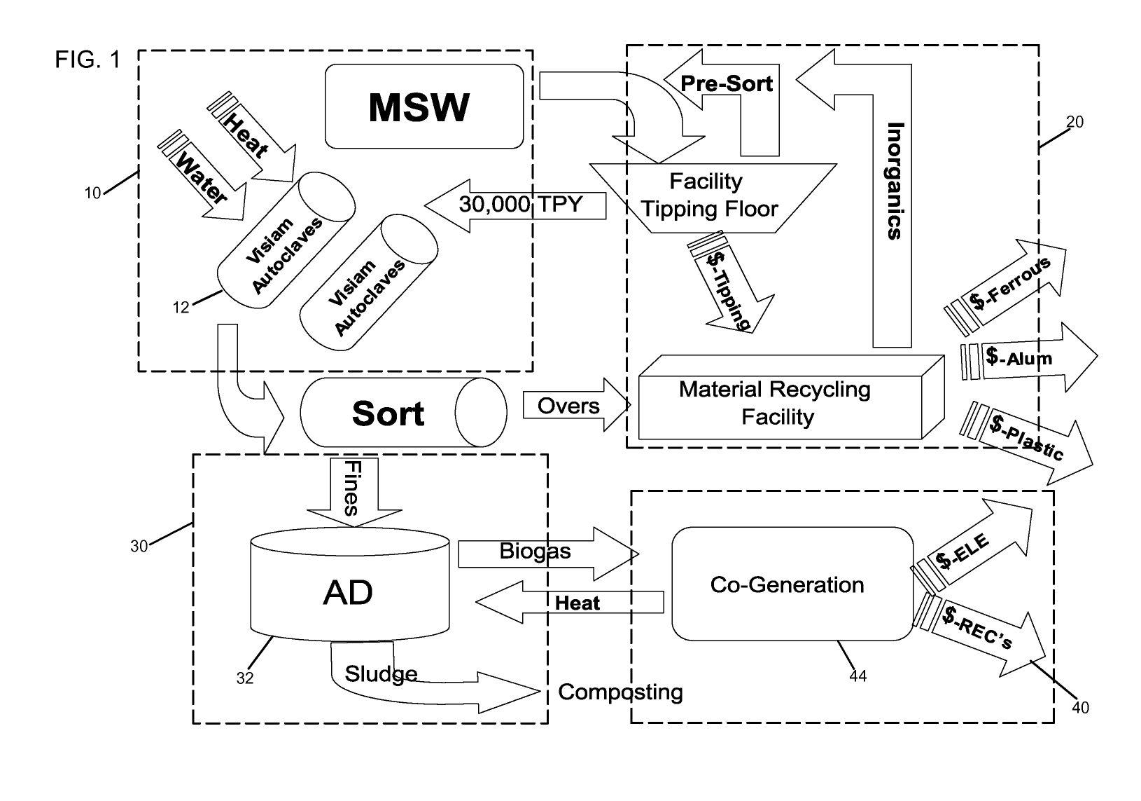

The system described herein (and illustrated in FIG. 1) comprises an integrated system, where the various different processes of pretreatment, sorting/separating, anaerobic digestion and conversion of biogas to fuel and electricity are fully integrated into a single process flow. Such integration improves the efficiency of conversion of waste to fuel, and recycling the heat generated by the system back into the single process flow conserves energy and improves the energy generation output of the system.

FIG. 1 illustrates a fully integrated system for processing solid waste. An example of solid waste is municipal solid waste (MSW). The term “municipal solid waste” means waste material, refuse, or garbage arising from residential locations, businesses, industrial sites, military sites, government sites and the like. Such waste includes cellulosic material, metals (both ferrous and non-ferrous), plastic, glass, food and others. Such waste can be derived from packaging materials that can be mixed cellulosic paperboard packaging materials, corrugated paperboard, plastic wrap, plastic bottles, steel cans, aluminum cans, other plastic or metal packaging materials, glass bottles, container waste and the like. Such waste can be any combination of plastic, metal, and paper. The systems described herein utilize the large fraction of MSW streams that are comprised of natural organic compounds (such as, for example, food and plant wastes). The organic fraction of municipal solid waste (OFMSW) is high in moisture content and has low heat value, and anaerobic digestion of the fraction can be used to produce methane gas, which is captured and used for energy cogeneration.

Material typically available in MSW streams can be used either as feed stock for fuel production or a source of recyclable material. MSW can also be combined with different types of organic feedstock, including, without limitation, plant waste, food waste, agricultural waste (such as corn stover, for example) and the like. The organic feedstock mixed with MSW streams may vary with season. MSW contains a wide variety of waste or discarded material. The material may include biodegradable and non-biodegradable waste, metal, paper, plastic, paints, varnishes, solvents, fabrics, wood material, glass, various types of chemical waste, pesticides and the like.

Organic materials available in municipal waste include, for example, cellulosic fiber or pulp, paperboard, corrugated paperboard, newsprint, glossy magazine stock, and a variety of other cellulosic board or sheet materials, including polymers, fillers, dyes, pigments, inks, coatings and a variety of other materials. Materials available in MSW streams also include natural organic compounds such as present in plant and food waste, such as for example, peat, hemp, jute, sugarcane, coconut husk, corn husk, rice hulls, wheat chaff, sewage sludge, wood fibers, paper fibers and the like. Recyclable materials in MSW include, without limitation, plastics, glass, ferrous metals, non-ferrous metals and other materials capable of being recycled. Plastics common in recyclable materials streams include polyolefins such as, for example, polyethylene, polypropylene, polyesters such as polyethylene terephthalate, polyvinyl chloride, mixed stream plastics and other thermoplastic materials. Metal streams include, for example, ferrous magnetic metals such as iron, steel and magnetic alloys, non-ferrous magnetic metals such as aluminum and other such materials in the form of cans, sheets, foils, etc. Glass material can be clear or colored (i.e. green or brown). Other types of solid waste not mentioned herewith can also be processed using the system and processes described herein. These include, for example, medical waste, manure, animal carcasses, and the like. Other forms of organic feedstock such as corn stover, for example, can be combined with MSW and be processed in the integrated system described herein. The MSW streams can be presorted to remove large pieces of waste from the integrated processing system, such as, for example, furniture, large animal carcasses, and the like.

The integrated system shown in FIG. 1 features a subsystem 10 for pretreating MSW streams, or streams containing MSW, organic feedstock and combinations thereof. In an embodiment, the subsystem 10 comprises one or more pressurized vessels 12 (designated as “vessels” in FIG. 1) for introducing heat and water into the MSW stream. In an aspect, the vessel 12 is a rotating vessel. In another aspect, the vessel 12 is configured to have various positions. The vessel can be, for example, in a raised, horizontal, charging (or loading) position during introduction of MSW streams into the vessel. The vessel can be operated, for example, in the raised horizontal position for pretreatment of MSW. When the pretreatment process is complete, the vessel 12 is lowered to a lower discharge position to remove the treated MSW and move the contents to other subsystems 20 and 30 for additional processing and conversion to fuel.

In the vessel 12, at appropriate conditions of temperature, pressure and humidity, and with the rotating mechanical action of the vessel, the MSW stream is partially transformed into a fibrous cellulosic mass, separable metals and other recyclable materials. The agitation of the vessel 12 combined with the changing temperature, pressure and humidity conditions in the vessel help break fiber-to-fiber bonds, and produce substantially increased fibrous character in the particular cellulosic material in the MSW stream. The change in pressure and change in temperature causes substantial changes in the nature of water within the fibrous material. The change of water from a liquid to steam improves the quality of the fibrous material resulting in a fiber that can be recycled to provide a pulp or a fiber or further processed to a high quality fuel.

The vessel 12 includes apparatus for heating the interior of the vessel, for the introduction of water into the vessel and for evacuating steam from the interior of the vessel to introduce moisture or change the humidity level during pretreatment of MSW streams. In an embodiment, the quantity of water added to the interior of vessel 12 is about 30% to about 55% of the first weight of MSW. In another embodiment, the amount of water added is at a ratio of 0.01 to about 0.8 parts of water per part by weight of MSW. Water is introduced into vessel 12 by pumping from a condenser tank attached to vessel 12 (not shown in FIG. 1). Similarly, heat is added to the vessel 12 in a number of ways. In an embodiment, an amount of heat based on the first weight of MSW is added for a predetermined amount of time, such as for example, no more than 350 BTUs/Lb, or about 275 BTUs/lb in a time not greater than 75 minutes. As a result, the MSW is at a temperature of not greater than 350° F., for example, about 220° F. to about 330° F., or about 265° F. to about 285° F., typically 270° F., at a pressure of about 5 to about 25 psig, over a period of about 30 minutes to about 210 minutes, or about 45-90 minutes, or about 60-75 minutes. Heat is introduced into the vessel 12 by means of a working fluid, such as an oil, for example, that circulates through a conduit in vessel 12. Additional details on the structure of vessel 12 and the process for pretreatment of MSW in vessel 12 are provided in U.S. Pat. No. 7,497,392 and WO/2008/010970, both incorporated herein by reference.

In an embodiment, the system for conversion of MSW to biogas and/or energy comprises a subsystem 20 for a separator 22 that sorts and separates the pretreated MSW into an organic fraction and a recyclable materials fraction. The organic fraction comprises without limitation, plant waste, food waste, homogenous cellulosic mass derived from paper and or wood waste products, and other organic fines, and mixtures thereof. Once separated from the pretreated MSW stream, the organic fraction of MSW (OFMSW) is conveyed to a subsystem 30 for further processing.

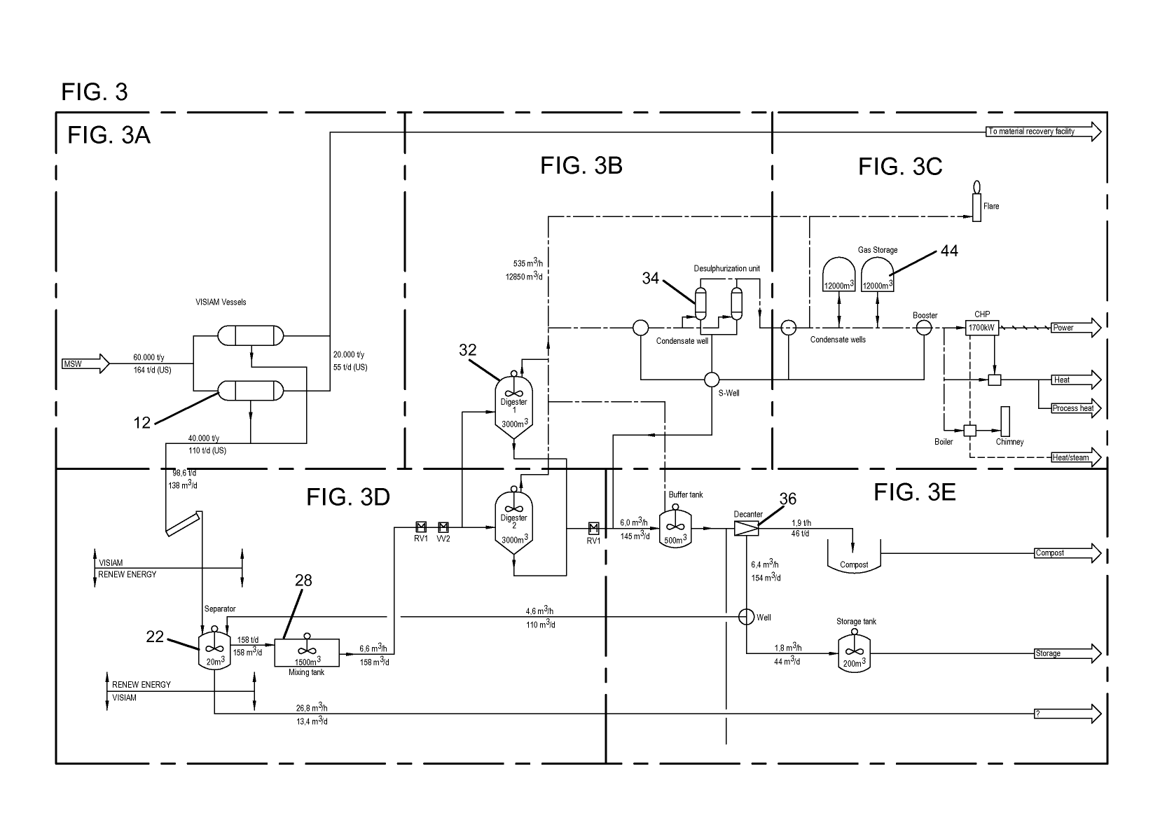

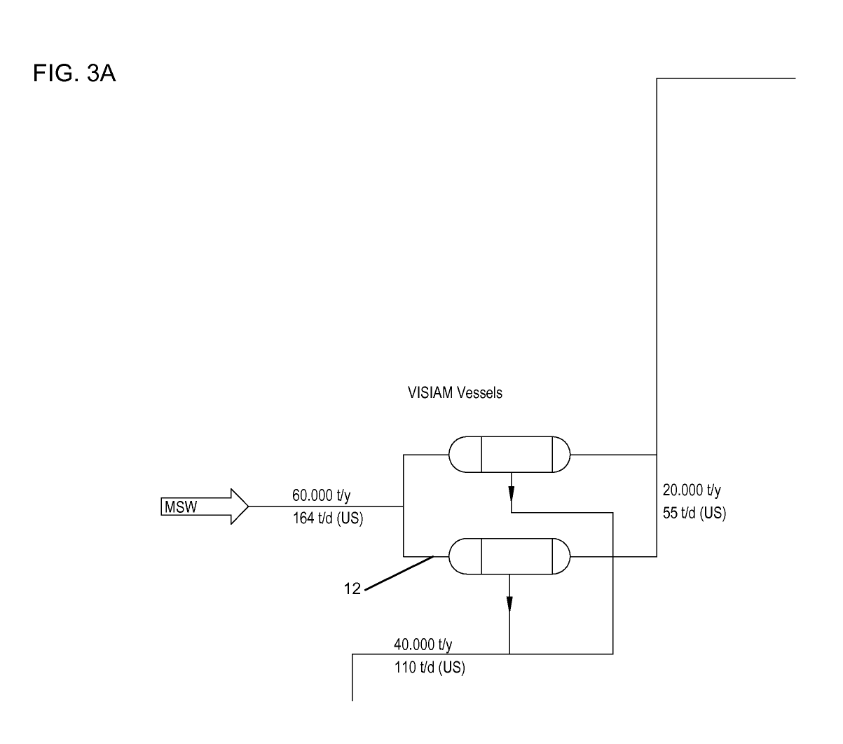

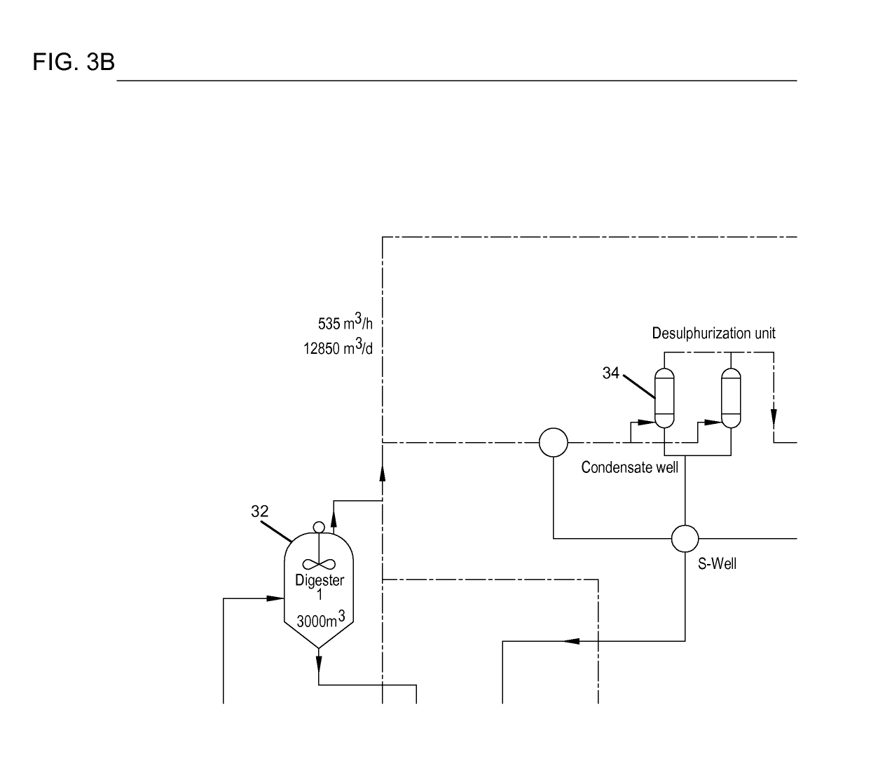

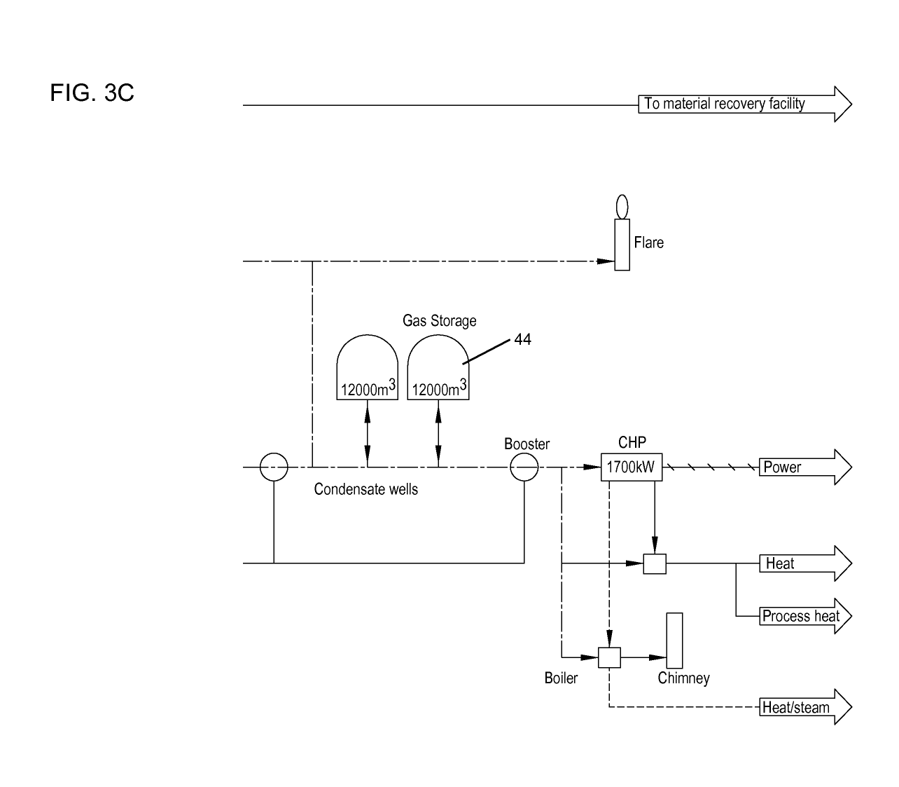

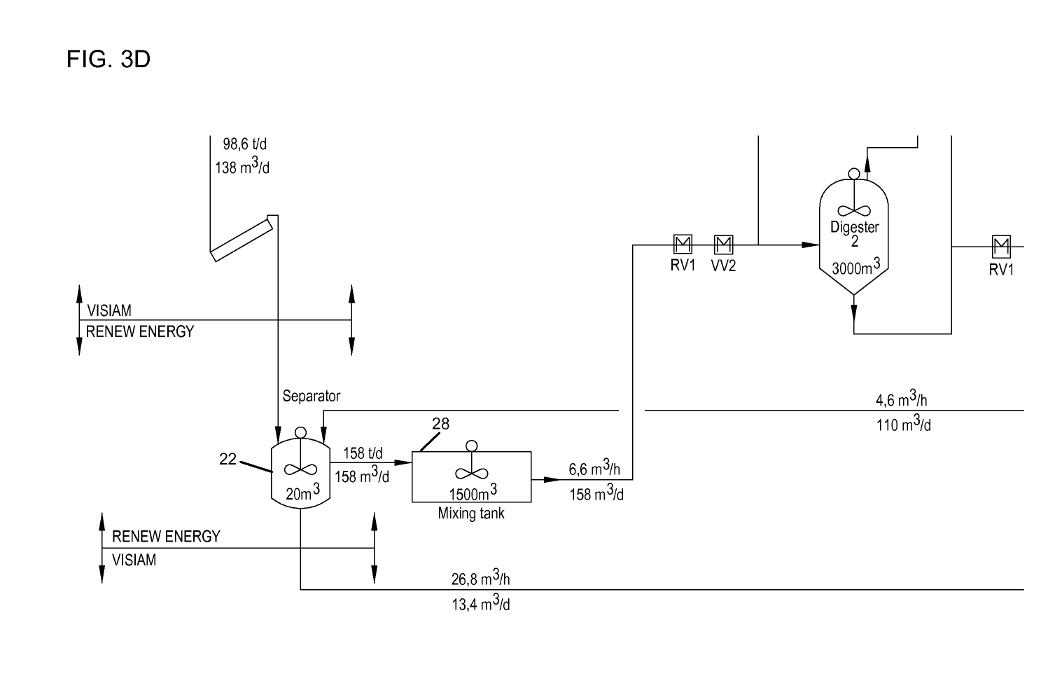

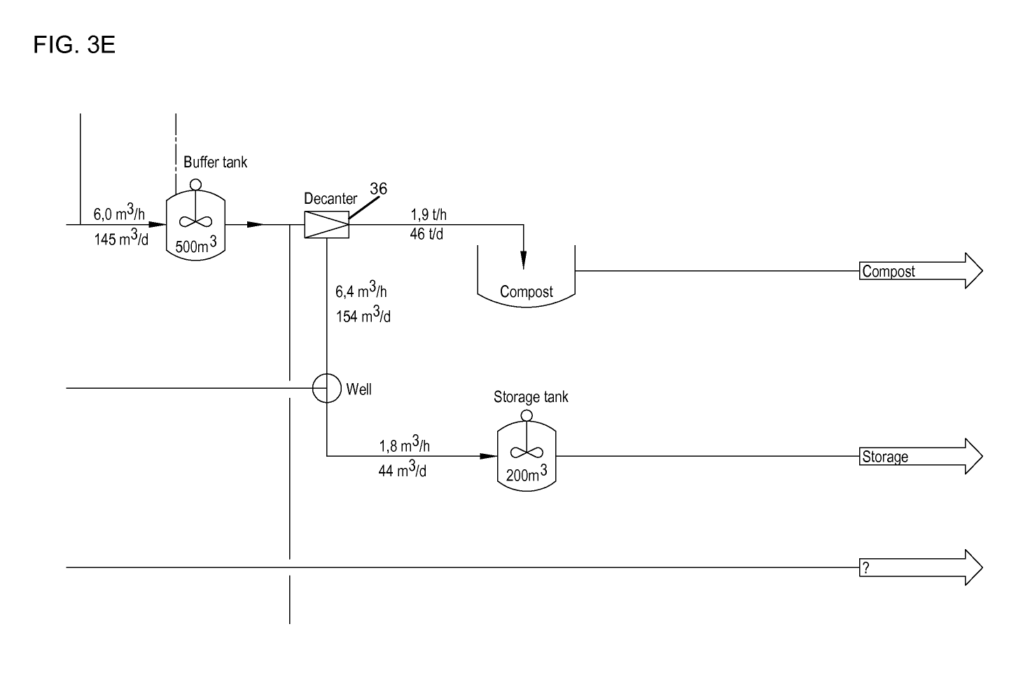

In an embodiment, the separator 22 (shown in FIGS. 1 and 3) comprises a mechanical device for sorting and separating large organic fractions from fine organic particulate matter, and also from recyclable materials. In an aspect, the mechanical device is a pulper which sorts organic material according to the size and weight of the material. The pulper sorts material such that only pieces small enough to pass through a trommel screen of a specific size become part of the homogenous organic fraction that will be conveyed to subsystem 30 for anaerobic digestion. In an embodiment, the trommel screen has a size of 19 mm, i.e. any material larger than this size will be excluded by the trommel screen. In an aspect, the pulper blends organic material with reject water from a decanter 36 used in sludge dewatering in subsystem 30.

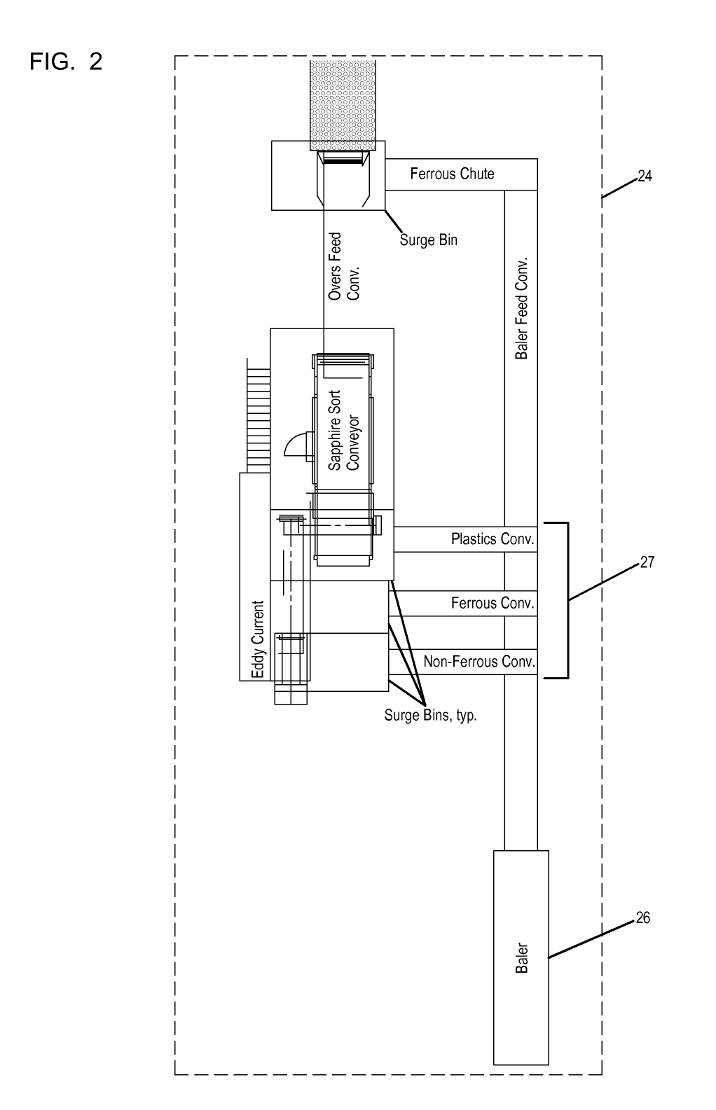

In an embodiment, the recyclable materials fraction of the pretreated MSW enters a materials recovery facility (MRF) 24 in a single stream. The single stream comprises a mixture of recyclable materials such as, for example, large pieces of glass, plastics, metals and some paper products (such as dense corrugated paper, for example). The MRF 24 then performs a gross sort of the single stream of pretreated MSW by type, i.e. ferrous metals, non-ferrous metals (such as aluminum, for example) and plastic. FIG. 2 is a schematic representation of MRF 24, which comprises a baler feed conveyor 25 used to convey plastics, ferrous and non-ferrous materials to a single baler 26. Surge hoppers 27 are needed to hold recyclable materials while other recyclables are conveyed to the baler. For example, when plastics are being sorted, the ferrous and non-ferrous materials would be held in surge hoppers 27 while the plastics are conveyed to baler 26.

In an embodiment, plastics separated from the MSW stream by MRF 24 can be subjected to pyrolysis to produce fuel in another part of subsystem 20. By “pyrolysis” is meant a recycling technique that converts plastic waste into fuels, monomers, or other valuable materials by thermal and catalytic cracking processes. It allows the treatment of mixed, unwashed plastic wastes. Thermal conversion leads the production of useful hydrocarbon liquids, such as, for example, crude oil, diesel fuel, and the like. Pyrolysis can be conducted at various different temperatures, with plastics pyrolysis generally carried out at a range of temperatures from low (less than 400° C.) to medium (400-600° C.) to high (above 600° C.), and is generally carried out at atmospheric pressure. Techniques of plastics pyrolysis are known to those of skill in the art, and are well described in Feedstock Recycling and Pyrolysis of Waste Products, J. Scheirs and W, Kaminsky, eds. (Wiley 2006). Pyrolysis is an endothermic process, and therefore, a supply of heat to the subsystem 20 is required, and this thermal requirement is met by heat generated within the integrated subsystem described herein.

In an embodiment, the subsystem 20 comprises a mixing tank. In the mixing tank, the OFMSW separated from the recyclable material in separator 22 is mixed with organic fines produced in the separating and sorting process. In an aspect, the OFMSW and organic fines mixture is combined with reject water from the decanter 36 (see FIG. 3), and the cellulosic mass formed is conveyed to the subsystem 30 for anaerobic digestion.

After sorting and separation of the pretreated MSW, the OFMSW is conveyed to a subsystem 30 (not shown in FIG. 1) for fermentation. By “fermentation” is meant a biological process by which sugars are converted into ethanol and carbon dioxide as metabolic products. Ethanol fermentation is an anaerobic process where yeast acts on the sugars in the feedstock in the absence of oxygen. The ethanol produced by this process can be used as fuel. Fermentation occurs in three steps of glycolysis, pyruvate formation, conversion of pyruvate to acetaldehyde, and reduction.

During glycolysis, the sugars in the OFMSW, namely glucose, fructose and cellulose, are broken down by the yeast into pyruvate, energy in the form of two molecules of NADH and water. The yeast is used as freely suspended yeast cells, and many different types of yeast can be used in ethanol fermentation, such as for example, Saccharomyces cerevisiae, S. pombe, S. pastorianus and the like. Other types of yeast that are used primarily in an anaerobic setting include, for example, Kluyveromyces lactis, K. lipolytica and the like. Of these, S. cerevisiae is the most commonly used form of yeast in ethanol production, and can be used in both aerobic and anaerobic conditions.

Following glycolysis, the pyruvate is converted into acetaldehyde and carbon dioxide by the action of enzymes, specifically the enzyme pyruvate decarboxylase. In anaerobic conditions, this enzyme starts the fermentation process by converting pyruvate into acetaldehyde and carbon dioxide. The enzymes uses two thiamine pyrophosphate (TPP) and two magnesium ions as cofactors. The acetaldehyde is then reduced to ethanol by the action of the NADH formed during glycolysis. The process of industrial fermentation of organic feedstock to produce fuel-grade ethanol is known to those of skill in the art. The integration of fermentation and ethanol production into the integrated system described herein improves the overall efficiency and yield of the system. For example, organic feedstock and/or MSW streams weighing about 2000 lbs. will produce approximately 120 gallons of fuel-grade ethanol.

Following fermentation, the remaining organic fraction (OFMSW), now comprised largely of proteins, is conveyed to a subsystem 40 (see FIG. 1) for anaerobic digestion in a digester 42 (see FIG. 3). By “anaerobic digestion” is meant a complex biochemical process where microorganisms act in the absence of oxygen, in aqueous and neutral pH conditions, to break down organic compounds into the ultimate end products of methane and carbon dioxide. Anaerobic digestion takes place in four steps of hydrolysis, acidogenesis, acetogenesis and methanogenesis, ultimately leading to the production of biogas (i.e. methane and carbon dioxide).

During hydrolysis, the particulate matter in the complex organic matter, namely the fibrous or homogenous cellulosic mass that makes up OFMSW, is hydrolyzed by the action of hydrolytic bacteria into soluble organic polymers, monomers or other components, such as carbohydrates, amino acids, glucose, fatty acids and glycerol, for example. Hydrolytic bacteria are thermophilic bacteria that produce extracellular enzymes such as, for example, cellulase, hemicellulase, amylase, lipase, protease and the like. These enzymes break down the OFMSW into soluble components such as sugars, fatty acids and amino acids, for example. These soluble components are then subjected to acidogenesis. An example of hydrolytic bacteria is a microorganism such as Thermoanaerobium brockii.

In acidogenesis, a group of microorganisms known as acidogenic (or acid-forming) bacteria ferment or convert the sugars and amino acids into their components, i.e. carbon dioxide, H₂S, hydrogen, ammonia and simple organic acids, such as acetic, propionic, formic, lactic, butyric or succinic acids, for example. Other fermentation products include alcohols (such as methanol, ethanol and glycerol, for example), ketones (such as acetone for example), and esters (such as acetate, for example). The products formed vary according to the type of bacteria used as well as conditions (namely temperature and pH). The hydrogen and acetate can be acted on by methanogenic bacteria to produce biogas, but the volatile fatty acids (i.e. those longer than acetate, such as propionic and butyric acids, for example) must first be catabolized by acetogenesis.

In acetogenesis, a group of microorganisms known as acetogenic bacteria or acetogens convert the volatile fatty acids formed during acidogenesis to acetic acid or acetates, along with additional ammonia, hydrogen and carbon dioxide. Acetogenic bacteria convert the longer-chain fatty acids (e.g., propionic acid, butyric acid) and alcohols into acetate, hydrogen, and carbonic acid, which are used by the methanogens to produce biogas. Acetogenic bacteria fall into three categories: homoacetogens, syntrophes and homoreductors. Examples of acetogenic bacteria include, without limitation, members of the Clostridium genus, including for example, C. aceticum, C. thermoaceticum, C. termoautotrophicum, C. formiaceticum and members of the Acetobacter genus, such as for example, A. woodii, and the like.

The final stage of anaerobic digestion involves methanogenesis, wherein the intermediate products from the acidogenesis and acetogenesis phases are converted into the end products of anaerobic digestion, namely biogas, or a mixture methane, carbon dioxide and water. Methanogenesis is carried out between pH 6.5 and 8. Any OFMSW that remains unprocessed or undissolved at the end of the anaerobic digestion (i.e. undigested OFMSW and bacterial residue from the digestion) is sludge.

In an embodiment, subsystem 40 includes a decanter 46 for thickening and dewatering of the sludge, i.e. undigested OFMSW (see FIG. 3). In an aspect, the decanter comprises mechanical means such as a centrifuge, for example. The centrifuge is used to increase drainage of water from the sludge to thicken it. In another aspect, application of vacuum pressure can be used for dewatering and thickening the sludge. In yet another aspect, chemical means can be used for dewatering and thickening the sludge. A combination of mechanical and chemical means is ideal for dewatering of sludge. Thickened and dewatered sludge is used as a compost material, or as a soil conditioner after the sludge has been cured. Reject water produced in the decanter 46 is recycled back into the mixing tank 28 of subsystem 20 wherein the water is used to mix OFMSW and organic fines separated from the pretreated MSW. In an embodiment, the sludge comprises only about 3% to about 10% of the total weight of MSW and/or organic feedstock first introduced into the integrated system.

In an embodiment, subsystem 40 includes means for pyrolysis of the undigested residue of OFMSW or sludge to produce fuel. In an aspect, pyrolysis of the sludge occurs by flash pyrolysis, where the sludge is quickly heated to temperatures between about 350° C. to about 500° C. for less than two seconds. In another aspect, hydrous pyrolysis is used, where superheated water or steam is used to treat the sludge and convert into fuel. In yet another aspect, pyrolysis is carried out under pressure at temperatures greater than 430° C., or between about 450° C. and about 550° C. Pyrolysis of the sludge produces fuel at high yield. For example, pyrolysis of about 2000 pounds of thickened and dewatered sludge will produce approximately 200 gallons of fuel as an end product.

In an embodiment, subsystem 40 comprises a desulphurization unit 44 (see FIG. 3), where digester off-gases from anaerobic digestion, such as H₂S, for example, are removed or reduced to very low levels. Sulfurous acids and alcohols formed as byproducts of anaerobic digestion are recycled back into the integrated waste processing system.

Anaerobic digestion is carried out at varying temperature ranges, determined by the nature of the bacteria used for the digest. Some anaerobic bacteria can be used at temperatures ranging from below freezing to above 135° F. (57.2° C.), but they thrive best at temperatures of about 98° F. (36.7° C.) (mesophilic) and 130° F. (54.4° C.) (thermophilic). Bacteria activity, and thus biogas production, falls off significantly between about 103° and 125° F. (39.4° and 51.7° C.) and gradually from 95° to 32° F. (35° to 0° C.). In a preferred embodiment, anaerobic digestion and production of biogas is carried out a temperature of 52° C.

In an embodiment, the subsystem 50 of the integrated system shown in FIG. 1 is used to store the methane gas formed by anaerobic digestion of the OFMSW. In an aspect, the subsystem 50 comprises a flare system 52 and a low energy fuel reciprocating engine generator 54 (as in FIG. 3), which are used to process methane gas produced in the anaerobic digestion of OFMSW. As methane is a greenhouse gas and is considered to have a higher global warming potential than carbon dioxide, the subsystem either combust the methane gas in the flare system or store the gas for use in the engine generator.

In an embodiment, the subsystem 50 comprises a flare system 52. By “flare system” is meant a system for use or disposal of excess gaseous fuel streams by combustion, and includes, for example, ground flares, flare stacks, and the like. In an aspect, the flare system 42 is used to combust excess low BTU methane or other combustion gasses produced by anaerobic digestion of OFMSW. In an aspect, the flare system 42 of subsystem 40 is automated to ensure that all excess methane that is present after digestion passes through the flare system and is combusted. In an aspect, the flare system 42 of subsystem 40 can include pressure control or flow control devices to maintain proper flow of biogas into the flare system for combustion of excess low BTU gas. The flare system 42 can also include a mechanism by which the flare is triggered. For example, a continuous ignition system (using sparking electrodes, for example) can be used such that methane combustion occurs whenever methane gas enters the flare system.

In an embodiment, the subsystem 50 comprises a low energy reciprocating engine cogenerator 54 for use of the stored methane gas formed by anaerobic digestion of OFMSW. The reciprocating engine cogenerator 44 of subsystem 40 comprises an internal combustion engine with a component for burning fuel and a reciprocating piston that helps generate energy. For example, if the engine is equipped with a reciprocating piston that includes a magnetic coil system, the engine can be used to produce electrical energy. Engine generators of this type are known to those of skill in the art.

In an embodiment, the reciprocating engine generator of subsystem 40 is part of an energy cogeneration unit or subsystem. When the supply of methane gas from the anaerobic digestion subsystem reaches a level high enough for operation of the engine generator, the cogeneration system becomes operational. By “level high enough” is meant an amount of methane gas that is high enough to match the heat requirements of the thermal vessel 12 during pretreatment of MSW, and the heat requirements of the anaerobic digestion subsystem 40 and the thermal requirements of pyrolysis of plastics and/or sludge left after fermentation and anaerobic digestion. Alternatively, the cogeneration system produces energy that is used only for electrical sales and supplements the heat requirements of the thermal vessel 12 and the anaerobic digestion subsystem 40. For example, the engine cogenerator could produce up to approximately 1700 kW of electricity, which can be supplied to a local utility grid.

In an embodiment, the reciprocating engine generator of subsystem 50 includes a heat recovery steam generator attached to the exhaust stack of the engine generator This heat recovery steam generator is used to recover waste heat produced by combustion of methane gas in subsystem 50. Recovered waste heat can be directed back to the plant's heating system, thereby offsetting natural gas consumption by the entire waste processing system. The use of heat recovery increases the efficiency of the engine generator from about 30% to near 70%, assuming complete heat recovery.

A method for processing municipal solid waste (MSW) and converting into fuel is described herein. In an aspect, the method comprises pretreating MSW, sorting and separating the organic fractions and the recyclable materials in the MSW stream, subjecting the organic fraction of MSW (OFMSW) to anaerobic digestion, and converting the products of anaerobic digestion into biogas for use as fuel and energy.

Referring to FIG. 3, a solid waste stream is introduced into one or more pressurized rotary vessels 12. A quantity of water is introduced and the pressure inside vessel 12 is reduced. The interior of vessel 12 is then heated, resulting in the breaking of fiber-to-fiber bonds in the cellulosic material of the MSW stream. The change in pressure and change in temperature causes substantial changes in the nature of water within the fibrous material. The change of water from a liquid to steam improves the quality of the fibrous material resulting in a fiber that can be recycled to provide a pulp or a fiber or further processed to a high quality fuel.

After pretreatment, the vessel 12 is evacuated and the pretreated MSW stream is conveyed to a materials recovery facility (MRF) 24 in a single stream, as shown in FIG. 3. The single stream comprises a mixture of recyclable materials such as, for example, large pieces of glass, plastics, metals and some paper products (such as dense corrugated paper, for example). The MRF 24 then performs a gross sort of the single stream of pretreated MSW by type, i.e. ferrous metals, non-ferrous metals (such as aluminum, for example) and plastic. FIG. 2 is a schematic representation of MRF 24, which comprises a baler feed conveyor 25 used to convey plastics, ferrous and non-ferrous materials to a single baler 26. Surge hoppers 27 are needed to hold recyclable materials while other recyclables are conveyed to the baler. For example, when plastics are being sorted, the ferrous and non-ferrous materials would be held in surge hoppers 27 while the plastics are conveyed to baler 26.

After sorting and separation of the pretreated MSW, the organic fraction (OFMSW) is conveyed to a subsystem 30 for fermentation (not shown in figures) and then subsequently to a subsystem 40 (see FIG. 1 and FIG. 3) for anaerobic digestion, after combining with other organic fines separated and sorted in subsystem 20 in mixing tank 24. The various biochemical processes involved in ethanol fermentation and anaerobic digestion are as described herein and known to those of skill in the art.

In an embodiment, the method for converting the OFMSW into fuel comprises converting the methane gas formed by anaerobic digestion into fuel. In an aspect, the method uses a flare system 52 and a low energy fuel reciprocating engine generator 54, to process methane gas produced in the anaerobic digestion of OFMSW. In an embodiment, the flare system is used to combust methane produced by anaerobic digestion of OFMSW. In an aspect, the flare system is automated to ensure that all biogas or methane that is present after digestion passes through the flare system and is combusted.

In an embodiment, the method for converting methane gas into fuel comprises using reciprocating engine cogenerator for storing methane gas formed by anaerobic digestion of OFMSW. Use of reciprocating engine generators for storage of methane gas for use as fuel are as described herein and known to those of skill in the art.

In an embodiment, the method for converting methane gas to fuel comprises using a heat recovery attached to the exhaust stack of the engine generator. This heat recovery steam generator is used to recover waste heat produced by combustion of methane gas in subsystem 50. Heat in the amount of approximately about 750 BTUs to about 1500 BTUs can be recovered using subsystem 50. Recovered waste heat can be directed back to the plant's heating system, thereby offsetting natural gas consumption by the entire integrated waste processing system. The use of heat recovery increases the efficiency of the engine generator from about 30% to near 70%, assuming complete heat recovery.

The systems and methods of the invention produce fuel having a typical heat value of at least 2500 BTU/lb at a moisture content of 55%. The heat value of materials is typically at or near the heat value for cellulose, and can be about 2500 BTU/lb to about 8500 BTU/lb, depending on the waste source and the moisture content. In typical MSW streams, the density of unprocessed waste is 15 lb/ft3, and a process time of not greater than 85 minutes, typically about 70-80 minutes, for example about 75 minutes. Typically, the converted mass has an overall volume that is not greater than 50% of the volume of the MSW stream before processing, typically about 33% (one third) the volume. In other words, the converted mass undergoes a volume reduction of about 50-66% after processing, relative to the initial volume of the MSW stream.

The above specification, examples and data provide a complete description of the manufacture and use of the composition of the invention. Since many embodiments of the invention can be made without departing from the spirit and scope of the invention, the invention resides in the claims hereinafter appended.

We claim:

1. An integrated waste processing system comprising:
(a) a first subsystem for pretreating municipal solid waste;
(b) a second subsystem for separating and sorting the pretreated municipal solid waste;
(c) a third subsystem for anaerobic digestion of the separated organic fractions; and
(d) a fourth subsystem for gas storage and cogeneration of energy.

2. The waste processing system of claim 1, wherein the first subsystem comprises one or more pressurized vessels for pretreating the municipal solid waste.

3. The waste processing system of claim 2, wherein the first subsystem comprises one or more pressurized vessels for addition of heat and water to the municipal solid waste.

4. The waste processing system of claim 1, wherein the second subsystem comprises a separator that separates the pretreated municipal solid waste into an organic fraction and a recyclable materials fraction.

5. The waste processing system of claim 4, wherein the organic fraction comprises a homogenous cellulosic mass.

6. The waste processing system of claim 4, wherein the recyclable materials fraction are sorted from a single stream.

7. The waste processing system of claim 6, wherein the sorted recyclable materials fraction comprises plastics, metal, paper products, fiber material and mixtures thereof.

8. The waste processing system of claim 7, wherein the sorted recyclable materials are further sorted into ferrous, non-ferrous, aluminum and plastic materials.

9. The waste processing system of claim 1, wherein the third subsystem comprises a system for thermophilic anaerobic digestion of organic fractions of the pretreated municipal solid waste.

10. The waste processing system of claim 9, wherein the system for anaerobic digestion comprises methane, carbon dioxide and a compost material as end-products.

11. The waste processing system of claim 1, wherein the fourth subsystem comprises a flare system and a low energy fuel reciprocating engine generator.

12. The waste processing system of claim 11, wherein the flare system and low energy fuel reciprocating engine generator are used to process methane formed by anaerobic digestion of the organic fraction of municipal solid waste.

13. The waste processing system of claim 11, wherein the flare system is used to combust the methane formed by anaerobic digestion of the organic fraction of municipal solid waste.

14. The waste processing system of claim 11, wherein the low energy fuel reciprocating engine generator is used to store methane gas.

15. The waste processing system of claim 14, wherein the low energy fuel reciprocating engine generator is used as part of a cogeneration energy system.

16. The waste processing system of claim 15, wherein the cogeneration energy system produces electricity.

17. The waste processing system of claim 13, wherein recoverable heat produced by combustion of methane is recovered by a heat recovery located on the low energy fuel reciprocating engine generator.

18. The waste processing system of claim 18, wherein heat recovery by the generator is used to offset natural gas consumption in plant heating systems.

19. The waste processing system of claim 18, wherein heat recovery by the generator improves the efficiency of the low energy fuel reciprocating engine generator from about 30% to about 70%.

20. A method for processing municipal solid waste comprising:
(a) pretreating municipal solid waste;
(b) sorting and separating the pretreated municipal solid waste into an organic fraction and a recyclable materials fraction;
(c) subjecting the organic fraction to anaerobic digestion; and
(d) converting one or more products of the anaerobic digestion into fuel.

21. The method of claim 20, wherein pretreating municipal solid waste comprises
(a) introducing the municipal solid waste into a rotary vessel;
(b) adding a quantity of water into the rotary vessel;
(c) reducing the pressure inside the rotary vessel;
(d) heating the interior of the rotary vessel; and
(e) evacuating the pretreated municipal solid waste in a single stream from the rotary vessel for sorting and separation.

22. The method of claim 20, wherein sorting and separating the pretreated municipal solid waste into an organic fraction and a recyclable materials fraction comprises:
(a) separating the organic fines from the pretreated municipal solid waste as the organic fraction; and
(b) sorting the recyclable materials by type.

23. The method of claim 22, wherein separating the organic fines from the pretreated municipal solid waste comprises separating food waste, plant waste, paper products, fiber material and mixtures thereof from the pretreated municipal solid waste.

24. The method of claim 22, wherein sorting the recyclable materials by type comprises separating the recyclable materials into ferrous metals, non-ferrous metals, aluminum, glass and plastic.

25. The method of claim 20, wherein anaerobic digestion of the organic fraction of municipal solid waste comprises:
(a) contacting the organic fraction with thermophilic bacteria;
(b) breaking down the organic fraction; and
(c) converting the digested organic fraction to biogas.

26. The method of claim 25, wherein the formed biogas comprises a mixture of carbon dioxide and methane.

27. The method of claim 20, further comprising using a flare to combust excess methane produced by anaerobic digestion.

28. The method of claim 20, wherein converting the formed biogas into fuel comprises storing the biogas for use in a reciprocating engine cogenerator.

29. The method of claim 27, wherein combustion of methane produces recoverable heat.

30. The method of claim 29, wherein recoverable heat is recovered using a heat recovery generator.

31. The method of claim 30, wherein recovered heat is used to offset natural gas consumption in plant heating systems.
